(19) United States
(12) Patent Application Publication
(10) Pub. No.: US 2005/0261149 A1
(43) Pub. Date: Nov. 24, 2005

(54) Reusable soap bar insert

(76) Inventor: Robert Lee Mekus, Redford, MI (US)

Correspondence Address: Robert Lee Mekus, 26826 Student, Redford, MI, 48239, US

(21) Appl. No.: 10/784,098

(22) Filed: Feb. 23, 2004

(51) Int. Cl. A61K 7/50

(52) U.S. Cl. 510/143

(57) ABSTRACT

The Reusable Soap Bar Insert is a device to be utilized with specially configured bars of bathing soap and is designed to help reduce soap waste. The new concept of this invention is that it is a non-disposable item that may be used again and again in new bars of soap, throughout soap usage. The soap would be manufactured with a hole through its length that is shaped to accommodate the Reusable Soap Bar Insert. Prior to using the bar of soap the user pushes the reusable insert into the pre-manufactured hole, where it stays until the soap is depleted. An optional lanyard may be attached to hang or handle the bar of soap more conveniently.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND OF THE INVENTION

After ordinary use of a bar of soap, a portion remains that is too small to handle conveniently and this remnant is often discarded. The invention helps to preclude this waste. In the patents identified below, an object is inserted into the soap bar during the manufacturing process. These inserts, having approximately the same volume as the usual remnant, would make more of the soap bar easily accessible to the consumer and thus reduce waste, however since the inserts are not reusable the economic viability is questionable and a survey shows that a comparable “reusable” product is not currently on the market.
1. Sawyer, No. Des 341,223 (Nov. 9,1993)—Described a soap bar with a foam insert.
2. Dunne, No. 648,247 (Apr. 24, 1900)—Advocated a core incorporated in the soap bar by the manufacturer. The core would contain advertising and provide buoyancy.
3. Stone, U.S. Pat. No. 1,997,474 (Apr. 9,1935)—Recommended a reinforcing core, porous and buoyant.
4. Bredice, U.S. Pat. No. 3,773,672 (Nov. 20, 1973)—Described an insert of solid rubber.
5. Lindauer et al, U.S. Pat. No. 4,438,010 (Mar. 20,1984)—Advocated a perfumed core.
6. Dulin, U.S. Pat. No. 5,221,506 (Jun. 22,1993)—Recommended a sponge-type structural insert.

### BRIEF SUMMARY OF THE INVENTION

The invention is a reusable insert for a bar of soap. This insert, provided by the soap manufacturer or distributor, is positioned by the consumer into a hole that the soap maker embodies in the bar of soap. The durable insert is retained and reused by the consumer.

With this configuration, soap usage is more efficient and the soap purchaser can conveniently use all of the material he pays for. The manufacturer requires fewer tools and facilities to produce the unique bar, since it contains less material.

This invention also makes it possible to utilize various accessories to facilitate soap handling and storage. An example is a lanyard or tether, however this component is not essential to the proper functioning of the invention.

### BRIEF DESCRIPTION OF THE SEVERAL VIEWS OF THE DRAWING

FIG. 1 is a side view of the Reusable Soap Bar Insert.

FIG. 2 is an end view of the Reusable Soap Bar Insert.

FIG. 3 is a cut-away view of the Reusable Soap Bar Insert in a bar of soap.

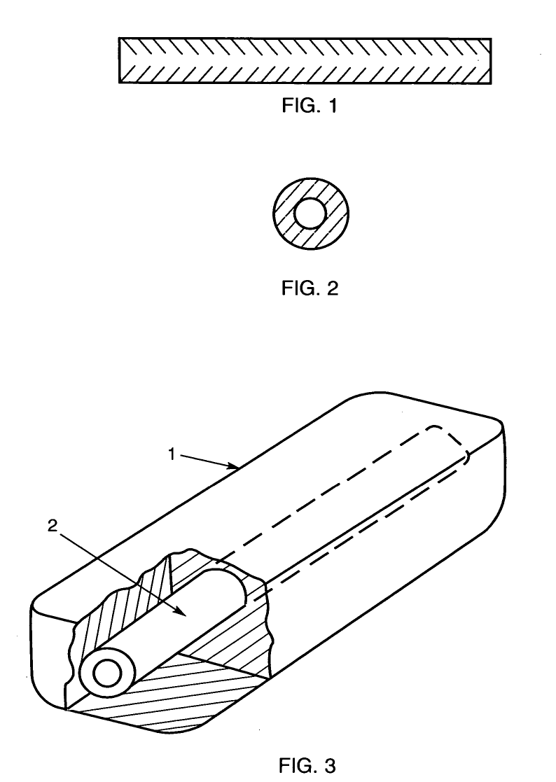

#1 is the provided bar of soap.

#2 is the insert.

### DETAILED DECRIPTION OF THE INVENTION

Two components are involved—a reusable insert and a uniquely configured bar of soap. The insert is composed of a durable, non-water soluble material such as plastic.

In one form the insert may be a hollow, round tube-like shape or a solid cylindrical shape, with the length being the same as that of the soap bar. Other geometric shapes, e.g. oval, are possible.

The soap bar has an unusual feature—a hole corresponding to the size and shape of the insert—centrally located in the bar. The hole and the insert have approximately the same diameter and length.

The exact dimensions and composition of these two components will be established by the manufacturer, and are dependant, for example, upon material chemistry and marketing strategy.

In actual practice, the two components are easily fitted together without special tools. When the soap is depleted, the use cycle can be repeated with a new bar of soap since the consumer retains the reusable insert.

Experiments confirm that the insert, occupying a central space in the bar, allow practically all of the soap to be conveniently accessible, thus reducing waste.

Since both components are produced or coordinated by the same source, normal quality control disciplines will ensure that the product dimensions and characteristics necessary for good performance will be adequately maintained.

It is expected that the two components would be available separately or together, from the manufacturer or distributor, according to the needs of the consumer.

Another benefit of the invention is that certain accessories can be utilized that will provide added convenience for the consumer. An example is the use of a lanyard or tether to facilitate use or storage of the soap.

1. A durable and reusable insert of a non-water soluble material such as, but not limited to plastic, to be positioned by the consumer in specially designed bar soap to reduce waste and spoilage.
